(19) United States
(12) Patent Application Publication
(10) Pub. No.: US 2018/0229299 A1
(43) Pub. Date: Aug. 16, 2018

(54) COPPER-SILICA CORE-SHELL NANOPARTICLES AND METHODS

(71) Applicant: THE BOARD OF TRUSTEES OF THE UNIVERSITY OF ARKANSAS, Little Rock, AR (US)

(72) Inventors: Cameron Crane, Hiwassee, AR (US); Jingyi Chen, Fayetteville, AR (US); Feng Wang, Fayetteville, AR (US)

(21) Appl. No.: 15/896,595

(22) Filed: Feb. 14, 2018

Related U.S. Application Data
(60) Provisional application No. 62/459,396, filed on Feb. 15, 2017.

(51) Int. Cl. B22F 1/02; B01J 23/72; B01J 21/08; B01J 35/00; B01J 37/02; C10M 125/04; C10M 125/26; A61K 41/00; A61K 49/00; A01N 25/28; A01N 59/20; G01N 21/552; G01N 21/65; B22F 1/00

(52) U.S. Cl. CPC B22F 1/02; B82Y 40/00; B01J 21/08; B01J 35/0006; B01J 35/0013; B01J 37/0221; C10M 125/04; C10M 125/26; A61K 41/0052; A61K 49/0002; A01N 25/28; A01N 59/20; G01N 21/554; G01N 21/658; B22F 1/0018; C10M 2201/05; C10M 2201/105; B01J 23/72

(57) ABSTRACT

In one aspect, compositions comprising copper-silica (Cu—SiO₂) core-shell nanoparticles are described herein. The core-shell nanoparticles comprise copper (Cu) core components and silica (SiO₂) shell components encapsulating the core components. In some embodiments, the nanoparticle compositions comprise a continuous aqueous phase and a population of copper-silica (Cu—SiO₂) core-shell nanoparticles dispersed in the aqueous phase.

### CROSS-REFERENCE TO RELATED APPLICATIONS

This application claims the benefit of U.S. Provisional Application No. 62/459,396 entitled “Copper-Silica Core-Shell Nanoparticles and Methods” filed Feb. 15, 2017, the contents of which are incorporated herein in their entirety.

### STATEMENT REGARDING FEDERALLY SPONSORED RESEARCH

This invention was made with Government support under Contract/Grant Nos. 145888 and 1563227, awarded by the National Science Foundation (NSF). The Government has certain rights in this invention.

### FIELD

This application relates to nanoparticles and, more particularly, to copper-silica core-shell nanoparticles and methods of making and using the same.

### BACKGROUND

Plasmonic metal nanostructures are gaining popularity in several tech industries for exhibiting a superior ability to manipulate light at the nanoscale, making them particularly useful for applications including, but not limited to, sensing, optical waveguiding, telecommunication, biomedicine, and plasmon-enhanced photocatalysis, among others. Several metals, including, but not limited to, silver (Ag), gold (Au), and copper (Cu), exhibit plasmonic resonances in the visible spectral range.

Cu is more earth abundant than Ag and Au, and thus particularly appealing as a low-cost plasmonic metal for practical applications. However, synthesizing Cu nanoparticles has proven problematic, because Cu nanoparticles are extremely prone to surface oxidation. When exposed to air, surface oxidation significantly broadens the LSPR peak and decreases the peak intensity of Cu nanoparticles. Formation of oxides (i.e., Cu₂O, CuO, and CuO_{0.67}) is difficult to prevent, especially for long periods of time. Preventing oxidation is a major challenge that has yet to be overcome for practical applications involving Cu nanostructures.

One approach aimed at reducing surface oxidation includes removing oxides post facto from the Cu nanostructures via oxide removing agents (e.g. acetic acid). This approach is problematic, as oxide removing agents can change the surface morphology of the nanostructures and degrade the optical properties. Another approach includes using polyvinylpyrrolidone (PVP) as a capping agent for Cu nanoparticles to limit oxidation. However, large amounts of PVP are required to reduce the oxidation rate and the method is not sustainable.

In view of these challenges, a need exists for improved nanoparticles.

### BRIEF DESCRIPTION OF THE FIGURES

The invention will now be described by way of example, with reference to the accompanying figures, of which:

FIG. 1A schematically illustrates copper-silica (Cu—SiO₂) core-shell nanoparticles and methods of making the same;

FIG. 1B is a transmission electron microscopy (TEM) image of Cu—SiO₂ core-shell nanoparticles; and

FIGS. 2A-9B show various characterization data for Cu—SiO₂ core-shell nanoparticles.

### DETAILED DESCRIPTION

Novel copper-silica (Cu—SiO₂) core-shell nanoparticles and methods of making and using the same are set forth herein. The implementations described herein can be more readily understood by reference to the following detailed description, examples, and drawings. The nanoparticles and methods described herein, however, are not limited to the specific implementations presented in the detailed description, examples, and drawings. It should be recognized that these implementations are merely illustrative of the principles of the present disclosure. Numerous modifications and adaptations will be readily apparent to those of skill in the art without departing from the scope of the instant disclosure.

In addition, all ranges disclosed herein are to be understood to encompass any and all subranges subsumed therein. For example, a stated range of “1.0 to 10.0” should be considered to include any and all subranges beginning with a minimum of 1.0 or more and ending with a maximum value of 10.0 or less, e.g., 1.0 to 5.3, or 4.7 to 10.0, or 3.6 to 7.

All ranges disclosed herein are also to be considered to include the end points of the range, unless expressly stated otherwise. For example, a range of “5-10,” “between 5 and 10,” or “from 5 to 10” should generally be considered to include the end points of 5 and 10.

Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

The Cu—SiO₂ core-shell nanoparticles and methods of making and using the same as described herein advantageously exhibit intense and sharp localized surface plasmon resonance (LSPR) in the visible spectral range. Organic capping molecules are adsorbed onto the external surfaces of preformed Cu nanoparticles prior to encapsulating the nanoparticles in a SiO₂ shell, which assists in preventing oxidation of the Cu during formation of the SiO₂ shell.

### I. Compositions Comprising Copper-Silica Core-Shell Nanoparticles

In one aspect, compositions comprising copper-silica (Cu—SiO₂) core-shell nanoparticles are described herein. The core-shell nanoparticles comprise copper (Cu) core components and silica (SiO₂) shell components encapsulating the core components. In some embodiments, the nanoparticle compositions comprise a continuous aqueous phase and a population of copper-silica (Cu—SiO₂) core-shell nanoparticles dispersed in the aqueous phase.

Turning now to the specific components of Cu—SiO₂ core-shell nanoparticles, the Cu components include Cu cores, also referred to as “Cu nanoparticles”, synthesized by reducing a Cu precursor in the presence of one or more surfactants, including a volume of organic capping molecules. The organic capping molecules can comprise trioctylphosphine (TOP), which acts as a capping agent to passivate the external surfaces of the Cu cores and preserve the desirable colloidal characteristics of the Cu cores during a subsequent sol-gel process in which the Cu cores are coated with and/or encapsulated in SiO₂ shell components. Further, TOP acts in place of and/or in conjunction with other capping agents, including but not limited to alkylamines (e.g. oleylamine (OLAM), etc.), which can be used during and/or after synthesis of the Cu cores to prevent irreversible aggregation.

The Cu core components in a population of Cu—SiO₂ core-shell nanoparticles can have an average core diameter from 10 to 100 nanometers (nm), or a subrange thereof (e.g., 10-50 nm, 20-30 nm, 25-35 nm, 30-40 nm, 50-80 nm, 30-80 nm, 50-100 nm, etc.). The particle size can be mediated during synthesis in the presence of a capping agent, such as TOP. In certain embodiments, the Cu core components in a population of Cu—SiO₂ core-shell nanoparticles have an average core diameter of about 30 nm.

Moreover, the Cu core components in a population of Cu—SiO₂ core-shell nanoparticles can be, but are not limited to, spherical. In certain embodiments, the core components are not spherical, as non-spherical core components can exhibit an improved optical spectrum having a narrow, sharp localized surface plasmon resonance (LSPR) peak in the visible spectral range. For example and in some embodiments, the Cu core components comprise cubes, rounded cubes (i.e., cubes having rounded corners), nanorods, or combinations thereof.

Notably, the TOP-passivated Cu cores can be controllably synthesized to have a targeted size and/or shape prior the subsequent synthesis of Cu—SiO₂ core-shell structures via a sol-gel process in water/oil (W/O) microemulsion. The use of TOP capping agents dramatically improves the stability of Cu cores in the sol-gel process, thereby preserving the optical properties of the Cu cores after the synthesis. During synthesis, and as described below, the thickness of the SiO₂ shells increases with increased reaction time; however, the shells remain accessible to solvent molecules such as water. Furthermore, the self-assembled TOP-capped nanoparticles suspended in toluene could be effectively disassembled during the sol-gel reaction to recover their optical properties of individual nanoparticles in aqueous solution. Thus, the compositions and methods herein further the use of plasmonic Cu nanoparticles in aqueous solutions for biological and catalytic applications.

Referring now to the SiO₂ shell components that encapsulate the Cu cores, such components can have an average shell thickness of about 20 nanometers (nm) or less, or a subrange thereof (e.g., 5-10 nm, 5-15 nm, 8-12 nm, 10-15 nm, 10-20 nm, etc.). The SiO₂ shell components are coated on and/or over the Cu cores in a W/O microemulsion environment, which facilitates a sol-gel reaction as described in detail further below. The thickness of SiO₂ shell increases with increased reaction time during the sol-gel reaction.

After synthesis of Cu—SiO₂ core-shell nanoparticles, the core-shell nanoparticles can be indefinitely preserved by freezing or freeze drying and dispersed in water at a later date with their optical properties intact. Alternatively, the Cu—SiO₂ core-shell nanoparticles can be dispersed in an aqueous solution and used immediately or stored for later use. The aqueous solution can be water or a solution that is not water (e.g., polar organic solvents such as acetone, ethanol, etc.). Notably, compositions of Cu—SiO₂ core-shell nanoparticles in an aqueous solution have an improved shelf life during which the particles undergo no observable change with respect to optical characteristics, even when dispersed in water at ambient conditions for one week, two weeks, three weeks, one month, or less than six months.

Turning now to the optical properties of compositions of Cu—SiO₂ core-shell nanoparticles, such compositions can exhibit an optical spectrum having a localized surface plasmon resonance (LSPR) peak in the visible spectral range. Where the Cu—SiO₂ core-shell nanoparticles have Cu cores that are cubes or rounded cubes, the LSPR peak can range from 560-600 nm. Where the Cu—SiO₂ core-shell nanoparticles are nanorods, the LSPR peak ranges from about 600-800 nm. The compositions maintain these LSPR peak ranges even after being stored for at least one month, and in some aspects, between 1-6 months.

In certain embodiments, Cu—SiO₂ core-shell nanoparticle compositions exhibit an optical spectrum having two localized surface plasmon resonance (LSPR) peaks in the visible spectral range. For example, where the Cu—SiO₂ core-shell nanoparticles include a combination of Cu cores that are substantially cubic (e.g., rounded cubes) and nanorods, a first LSPR peak ranges from 560-595 nm and a second LSPR peak ranges from 600-800 nm. The first LSPR peak is characteristic of Cu—SiO₂ core-shell nanoparticles having substantially cubed or rounded cube shaped Cu cores and the second LSPR peak is characteristic of Cu—SiO₂ core-shell nanoparticles having nanorod shaped Cu cores.

The Cu—SiO₂ core-shell nanoparticles described herein can be disposed in compositions including, for example and not limited to, antimicrobial formulations, heterogeneous catalysts, tribology fillers, tribology compositions, photothermal agents, detection reagents for a Surface Enhanced Raman Spectroscopy (SERS) system and/or a SERS detector, and medical imaging contrast agents. For example, the Cu—SiO₂ core-shell nanoparticles described herein can be used as photothermal agents to absorb light and convert the light to heat for disease treatment or photocatalysis. The Cu—SiO₂ core-shell nanoparticles described herein can be used for many different applications across many different industries.

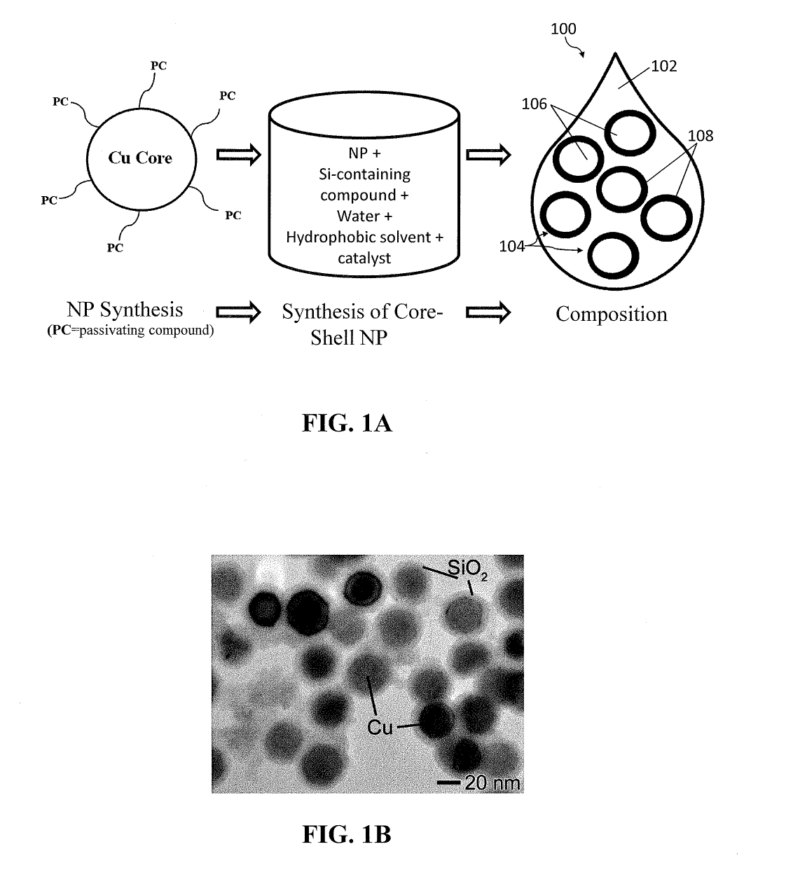

FIG. 1A schematically illustrates Cu—SiO₂ core-shell nanoparticles and methods of making the same according to some embodiments. Initially, Cu nanoparticles can be synthesized to include a Cu core that is passivated with a passivating compound PC, such as TOP. In some embodiments, a Cu—SiO₂ core-shell nanoparticle composition 100 comprises an aqueous solution 102 and a population of core-shell nanoparticles 104 dispersed in the aqueous solution. The core-shell nanoparticles 104 comprise Cu core components 106 (i.e., Cu cores) and SiO₂ shell components 108 encapsulating the core components. FIG. 1B is TEM micrograph of a population of Cu—SiO₂ core-shell nanoparticles. The dark spheres, appearing as circles in FIG. 1B, are Cu cores that are surrounded by SiO₂ which appears as a lighter contrasting layer surrounding the cores. Higher resolution TEM images illustrate the Cu cores as comprising a substantially cube shape, such as a rounded cube shape.

FIGS. 1A and 1B are for exemplary purposes only, as persons having skill in the art will appreciate that numerous modifications and adaptations are apparent, including modifications to the nanoparticle sizes and/or shapes, without departing from the instant disclosure.

### I. Methods of Making Copper-Silica Core-Shell Nanoparticles

According to another aspect of the instant subject matter, methods of making Cu—SiO₂ core-shell nanoparticle compositions are provided. Such a method comprises providing a population of Cu nanoparticles (Cu cores) having organic capping molecules adsorbed onto surfaces thereof and forming SiO₂ shells over the Cu nanoparticles. Exemplary organic capping molecules include TOP, a surfactant exposed to the Cu cores prior to and/or during formation of the SiO₂ shells.

In some embodiments, the population of Cu nanoparticles is a population of preformed Cu nanoparticles, meaning that the Cu cores are controllably synthesized prior to the reaction that forms the SiO₂ shells so that the preformed Cu cores have a desired size, shape, and/or a the cores have a range of a specific sizes and/or shapes. During a subsequent W/O microemulsion process, SiO₂ shells can condense over the nanoparticles and coat the exterior surface(s) of the preformed Cu nanoparticles via a sol-gel reaction that occurs during the W/O microemulsion process. The SiO₂ shells can at least partially encapsulate the Cu cores or fully encapsulate the Cu cores. For example, a SiO₂ shell can encapsulate at least 50% of a Cu core, at least 60% of the Cu core, at least 80% of the Cu core, at least 90% of the Cu core, or 100% of the Cu core. A population of core-shell nanoparticles can include only fully encapsulated cores, only partially encapsulated cores, or combinations of fully and partially encapsulated cores.

Further, methods of making Cu—SiO₂ core-shell nanoparticles can further comprise freezing the Cu—SiO₂ core-shell nanoparticles, dispersing the nanoparticles in an aqueous solution, and optionally freezing the nanoparticles in the aqueous solution. The optical properties and LSPR characteristics of the nanoparticles can be preserved, even if not frozen, for at least one month in an aqueous solution, or between about 1-6 months.

Additionally, a population of core-shell nanoparticles can comprise the reaction product of Cu nanoparticles having a particle size of 20 to 40 nm having organic capping molecules adsorbed onto surfaces of the Cu nanoparticles and a microemulsion formed from a hydrophobic solvent, water, a silicon (Si) containing compound, and an alkali hydroxide catalyst. The hydrophobic solvent can include a surfactant, fatty acid, or oil. The alkali hydroxide catalyst can comprise sodium hydroxide (NaOH) or potassium hydroxide (KOH). Notably, the reaction forming the nanoparticles and/or the nanoparticle compositions is devoid of ammonia, which has proven to degrade surfaces of the Cu cores. Stated differently, the microemulsion forming SiO₂ shells is devoid of ammonia.

FIG. 1A schematically illustrates a method of making a Cu—SiO₂ core-shell nanoparticle composition. For example, Cu nanoparticles can initially be preformed during nanoparticle synthesis. The synthesized Cu nanoparticles include a Cu core that is passivated with a passivating compound PC, such as TOP. The passivated surfaces of the Cu core resist oxidation.

In a subsequent step according to some embodiments, the synthesized nanoparticles are exposed to a Si-containing compound during a W/O microemulsion process. In the microemulsion process, the preformed Cu nanoparticles are mixed with a hydrophobic solvent, a Si-containing compound, water, and a catalyst during which SiO₂ shell components are formed over the Cu nanoparticles according to a sol-gel reaction. It is understood that FIG. 1A is for illustration purposes only, and persons of ordinary skill in the art will appreciate that the method can be altered so that two or more steps are combined, integrated, and/or additional steps can be added and such adaptations are apparent, without departing from the instant disclosure.

Some embodiments described herein are further illustrated in the following non-limiting examples. Chemicals employed in the following examples include Copper (II) 2,4-pentanedionate (Cu(aca)₂, 98%), 1-octadecene (ODE, 90%), Tetraethoxysilane (TEOS, 98%), Tri-n-octylphosphine (TOP, 90%), and sodium hydroxide (NaOH, 98%), purchased from Alfa Aesar. Oleylamine (OLAM, 70%) and poly-(oxyethylene) nonylphenyl ether (Igepal CO-520) were purchased from Sigma-Aldrich. Hexanes (ACS grade) and formic acid (99%) were purchased from EMD. Sulfuric acid (ACS grade, 98%) was purchased from BDH. All experiments were performed using 18 MΩ H₂O unless otherwise specified. All chemicals were used as received.

Transmission electron microscopy (TEM) images in the following examples were captured using a transmission electron microscope (JEOL JEM-1011) with an accelerating voltage of 100 kV. X-ray powder diffraction (XRD) was performed using a bench-top x-ray diffractometer (Rigaku Miniflex II). The hydrodynamic diameters of the products were measured using a dynamic light scattering (DLS) instrument (Brookhaven ZetaPALS). The concentration of Cu was determined using a flame atomic absorption (AA) spectrometer (GBC 932). UV-vis spectra were taken on a UV-vis spectrophotometer (Agilent Cary 50).

Further, the optical properties in the following examples were calculated according to the discrete dipole approximation (DDA) using the DDSCAT 7.3 program. In this formalism, the structure is represented by an array of dipole moments residing within its volume. Each volume element is represented as a dielectric continuum with the complex dielectric response function of bulk Cu. The optical cross sections were averaged over the two orthogonal polarization directions of the incident light. The optical efficiency, Q, is reported as the ratio of the respective optical cross section to π·a_{eff}², where the effective radius, a_{eff}, is defined as the radius of a sphere whose volume is equal to that of the structure. Optical spectra, including extinction, absorption, and scattering were simulated for each structure in water.

### Example 1

### Synthesis of Cu Nanoparticles

Cu nanoparticles (Cu cores) were synthesized by reducing a Cu precursor in the mixture of ODE and OLAM in the presence of CO. For example, Cu(acac)₂ (52.4 mg, 0.2 mmol) was added in the mixture of 4 mL ODE and 1 mL OLAM in a 25-mL 3-neck round bottom flask equipped with magnetic stirring and connected to a water-cooled condenser.

Ar gas was used to displace air and protect the mixture in the reaction flask prior to the addition of 1 mL TOP. The TOP capping passivates the surface of the Cu cores and protects the cores from oxidation and aggregation during the sol-gel process described in Example 2 further below. While maintaining the Ar protection, the reaction mixture was heated to 220° C.

To produce cube shaped Cu nanoparticles or cores, a small amount of carbon monoxide (CO, ˜15 mL) generated in situ from the dehydration of formic acid by sulfuric acid was flown over the hot reaction solution at 200° C. The vapor also contained formic acid due to its high vapor pressure. In this synthesis, formic acid acted as a reducing agent to accelerate the reduction while CO served as a capping agent to aid the formation of cubic nanoparticles. To produce mixtures of cube and rod shaped Cu nanoparticles or cores, CO was introduced by flowing the vapor over the reaction from 140° C. to 200° C.

After the reaction proceeded at 220° C. for another 20 min, the reaction was quenched by removal of the heating mantle and was allowed to cool to room temperature. Ethanol/toluene at a 4:1 ratio (25 mL) was added to the reaction mixture prior to centrifugation at 7800 rcf for 5 min to remove excess Cu precursor and surfactants. After discarding the supernatant, the pellet was dispersed in toluene for future use.

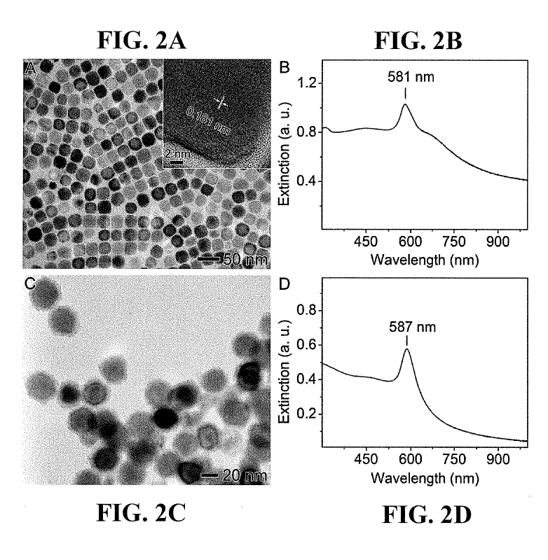

FIG. 2A shows a representative TEM image of Cu nanoparticles forming cubic Cu cores. In FIG. 2A, the cores have an average edge length of 28±3 nm. The inset of FIG. 2A is a HRTEM of an individual nanoparticle with the lattice spacing corresponding to the d spacing of the {200} planes of the face-centered cubic (fcc) structure of Cu. As FIG. 2B illustrates, these nanocubes suspended in toluene exhibited an LSPR peak at 581 nm with a shoulder at ˜700 nm. The SiO₂ shells were then coated on the surface as described in Example 2 below.

### Example 2

### Synthesis of Cu—SiO₂ Core-Shell Nanoparticles

The Cu—SiO₂ core-shell nanostructures were synthesized by modifying the established W/O microemulsion method set forth in the publication entitled “Mask-Assisted Seeded Growth of Segmented Metallic Heteronanostructures”, available in the Journal of Physical Chemistry at J. Phys. Chem. C 2014, 118, 28134-28132, the disclosure of which is hereby incorporated herein by reference, in the entirety.

In one embodiment, 1.2 mL Igepal CO-520 was added to 20 mL hexane in a 50-mL round bottom flask, followed by addition of the preformed Cu nanoparticles described in Example 1 above (˜8 pmol or ˜1 mg) and 30 μL TEOS, a Si-containing compound. The sol-gel reaction was catalyzed by adding 140 μl of 20 mM NaOH. The reaction was then allowed to precede either ˜24 h to produce thin SiO₂ shells or >48 h for thicker SiO₂ shells. After the reaction completed, equal volume of ethanol was added the reaction mixture, followed by centrifuging at 12,000 rcf for 30 min to collect the product.

The product was further purified with 30 mL ethanol twice, and collected by centrifuging at 12,000 rcf for 30 min or until a solid pellet was formed. The pellet was dispersed in water for further use.

The resulting core-shell nanostructures were imaged by TEM as shown in FIG. 2C. The shell thickness measured 4.6±0.8 nm, which can increase or decrease depending on the reaction time. The optical spectrum of the core-shell nanostructures was measured by UV-vis spectroscopy as shown in FIG. 2D. Notably, the aqueous suspension of these cubic core-shell nanostructures displayed a sharp LSPR peak at 587 nm, which, when compared to FIG. 2B illustrates preservation of the optical characteristics and LSPR of the Cu cores coated with SiO₂. The narrow and intense spectrum also indicated that the core-shell nanostructures are well dispersed in aqueous solution.

### Example 3

### Characterization and Simulation Data

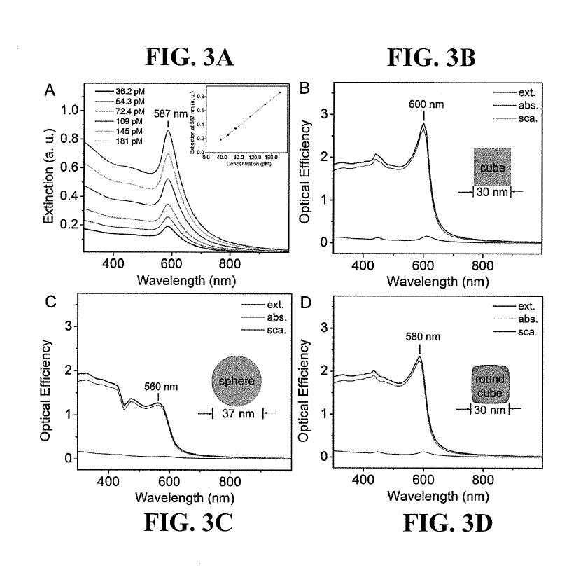

The LSPR of the Cu—SiO₂ core-shell structures were further quantified by fitting to the Beer-Lambert Law. The optical spectra of the core-shell structure aqueous suspensions were taken at a series of dilutions as shown in FIG. 3A. In FIG. 3A, the UV-Vis spectra of the Cu-SiO₂ core-shell nanoparticles is shown for different concentrations. The inset is the plot of the extinction at LSPR maxima as a function of concentration. The line in the inset is the linear fit of the data (y=4.77×10⁹×, R²=0.9998). That is, the absorbance at the LSPR maximum (587 nm) was plotted as a function of particle concentration and shown in the inset. Based on Beer's Law, the extinction coefficient of the suspension was determined to be 477×10⁹ M⁻¹ cm⁻¹ from the linear fit of the plot. From the extinction coefficient and the average size of the nanocubes (i.e. edge length of 28 nm), the extinction cross section of an individual nanocube was estimated to be 7.92×10⁻¹⁶ m². To further analyze the optical properties, the optical spectra for individual nanoparticles suspended in water were calculated using the DDA method.

FIG. 3B is the DDA simulated spectra of an isolated Cu nanocube with an edge length of 30 nm. The inset is the schematic drawing of the cross section of the nanocube. FIG. 3C is a DDA simulated spectra of an isolated Cu nanosphere with the same volume as the nanocube in FIG. 3B. The inset is the schematic drawing of the cross section of the nanosphere. FIG. 3D is a DDA simulated spectra of a rounded Cu nanocube with ˜2% of the total mass of the nanocube in FIG. 3B removed from the corners. That is, FIG. 3D is the DDA simulated spectra of a Cu core having a rounded cube shape. The inset shows the schematic drawing of the rounded nanocube viewed from the front. All simulated spectra were calculated using water as the medium.

Initially, the DDA simulation was only performed using two geometries with the same volume, i.e., the cube in FIG. 3B with edge length of 30 nm and the sphere in FIG. 3C with diameter of 37 nm. The size of the nanoparticle cubes can be varied to 80 nm or even 100 nm, where desired. The cube exhibits an extinction peak at 600 nm while the sphere shows an extinction maximum at 560 nm. In both cases, the extinction spectra were dominated by the absorption component. Notably, the optical efficiency of the cube is about twice as high as the sphere with the simulated extinction cross section being 3.06×10⁻¹⁵ m² and 1.38×10⁻¹⁵ m², for the cube and sphere, respectively. Although there is a small difference between the experimental and simulated peak position and cross section, the differences are explained by the slightly different sizes, shape, and the approximate nature of the experimental concentration measurement.

It is anticipated that the shape deviations of the synthesized nanocube from a perfect cube with sharp corners have the large influence on both peak position and intensity. According to TEM characterization in FIG. 2C, the synthesized nanocubes lost part of their mass from the corner regions, and thus do not have sharp corners as in a perfect cube. Therefore, DDA simulations were also performed on rounded cubic targets by removing up to 2% of the total mass from a perfect cube at the corner regions. The spectra of the rounded cubes is shown in FIG. 2D, where it can be seen the peak position shifted by 14 nm when the sharp corners were removed. The rounded cube has an extinction maximum close to 586 nm and is thus is in agreement with the experimental data in FIG. 3A.

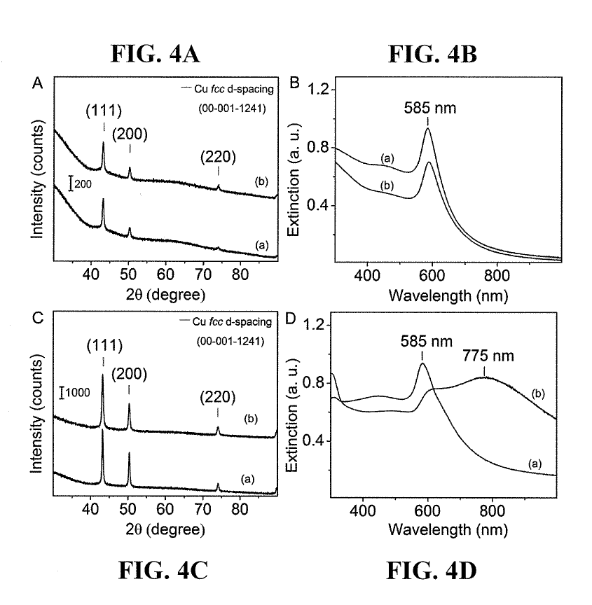

The stability of the core-shell nanostructures suspended in an aqueous solution was also characterized. FIG. 4A is XRD patterns for the Cu—SiO₂ core-shell nanostructures suspended in aqueous solution, FIG. 4B is UV-vis spectra for the Cu—SiO₂ core-shell nanostructures suspended in aqueous solution, and FIGS. 4C and 4D are characterization of as-synthesized Cu nanocubes suspended in toluene. The stability was assessed by both structural analysis using XRD and optical spectra using UV-vis. The XRD patterns of the Cu—SiO₂ core-shell nanoparticles were obtained before and after being stored for one month in aqueous solution as samples (a) and (b) in FIGS. 4A-4D, respectively. That is, in all panels, (a) and (b) represent the samples before and after 1-month storage, respectively.

Referring to FIG. 4A, the peaks at 43.3, 50.4, and 74.0 degree were indexed to {111}, {200}, and {220} planes of the face-centered cubic (fcc) Cu, confirming that the core-shell nanostructures contain pure Cu. There is no change in the XRD patterns in sample (b) when compared to sample (a), suggesting that no obvious oxidation occurred in the core-shell nanostructures during the one month storage.

The UV-vis spectra showed a sharp LSPR peak of Cu at 585 nm and remained unchanged before and after storage as shown in FIG. 4B. These results indicate that the Cu cores are well protected by the SiO₂ shell from oxidation and agglomeration.

The use of TOP-capped Cu nanoparticles facilitated the successful synthesis of well-dispersed Cu—SiO₂ core-shell nanoparticles with sharp and stable LSPR. The stability of TOP-capped nanoparticles of Cu by the XRD and UV-vis analysis before and after stored for a month in toluene. The XRD patterns before and after the storage were essentially the same, indexed to fcc Cu, showing no peaks from oxides. However, significant changes were found in the optical spectra of TOP-capped nanoparticles in toluene between before and after storage as shown in FIG. 4D. The initial sharp extinction peak at 585 nm red-shifted to ˜600 nm with reduced intensity and a broad shoulder centered at 775 nm arose in the optical spectrum after the TOP-capped nanoparticles were stored for a month. Since the XRD results showed no sign of oxidation, the spectral change is likely due to the particle agglomeration in solution.

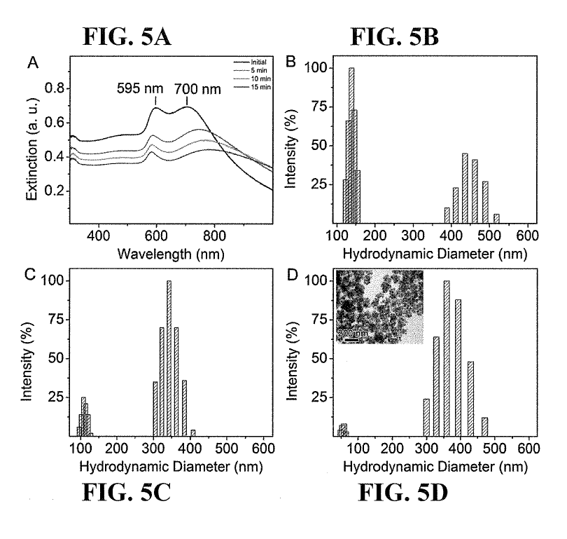

It is known that the agglomeration of TOP-capped nanoparticles is much more severe without sonication. The spectral changes of TOP-capped nanoparticles in toluene was monitored over 15 min at a 100-fold dilution (˜100 pM) compared to the typical concentration for storage in FIG. 5A. FIGS. 5B-5D is DLS data of the corresponding sample in FIG. 5A measured at the time of initial, 5 min, and 15 min.

Two peaks, 595 and 700 nm, were observed in the initial spectrum in FIG. 5A, which was taken 1 h after the reaction was completed. The 590-nm peak is attributed to individual nanoparticles while the 700-nm peak is attributed the nanoparticle agglomerates. Over the course of 15 min after dilution, the 700-nm peak became broader and shifted to the red to ˜800 nm, suggesting an increased number of agglomerates. These results were verified by the DLS measurements on the corresponding time course sample in FIGS. 5B-5D.

Two populations with different hydrodynamic diameters (HD) were observed: the one with HD<100 nm assigned to individual nanoparticles and the other with HD between 300-500 nm was assigned to nanoparticle agglomerates. By comparing FIGS. 5B-5D, the relative percentage of nanoparticle agglomerates was observed to increase over time. The nanoparticle agglomeration was further confirmed by TEM as shown in the inset of FIG. 5D. Each particle cluster seen in the TEM images contains about 10-20 TOP-capped nanoparticles. The agglomeration of TOP-capped nanoparticles stems from the hydrophobic interaction among long carbon chains of the TOP molecules attached to the particle surface in toluene.

Although TOP can protect the capped Cu nanoparticles from oxidation, the TOP-capped nanoparticles tend to agglomerate in toluene. Once the agglomerates form in toluene, it is difficult to separate into individual nanoparticles using mechanical methods, such as sonication. This tendency led to investigation of the effect of agglomeration on the formation of the core-shell nanostructures.

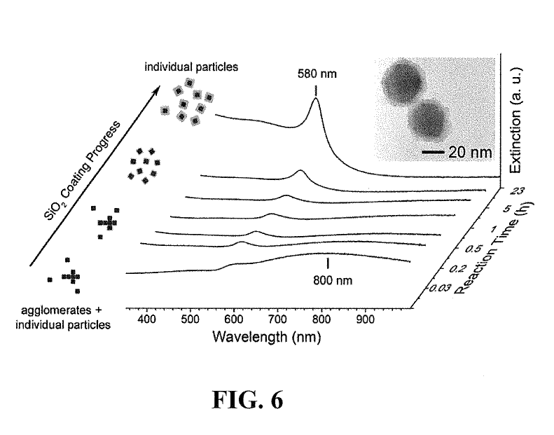

The SiO₂ coating process of the TOP-capped nanoparticle agglomerates was monitored over time using UV-vis spectroscopy. FIG. 6 is the spectral evolution of the time-course study on the SiO₂ coating process of the Cu nanoparticles.

Referring to FIG. 6, the extinction spectra of the aliquots taken from the reaction mixture during the sol-gel reaction is shown. During the SiO₂ coating that occurs during the W/O microemulsion process, the broad peak at ˜800 nm indicative of particle agglomeration gradually decreased and eventually disappeared as the reaction progresses to completion. The peak at 580 nm increased in intensity and became prominent as the reaction proceeded. This spectral evolution suggests that the agglomerates can be completely disassembled in the microemulsion and thus the SiO₂ coats on individual Cu nanoparticles rather than agglomerates. In this case, the surfactant Igepal CO-520 in the microemulsion disrupts the hydrophobic interaction between the carbon chains of TOP and aids the disassembly of the nanoparticles in the reaction mixture.

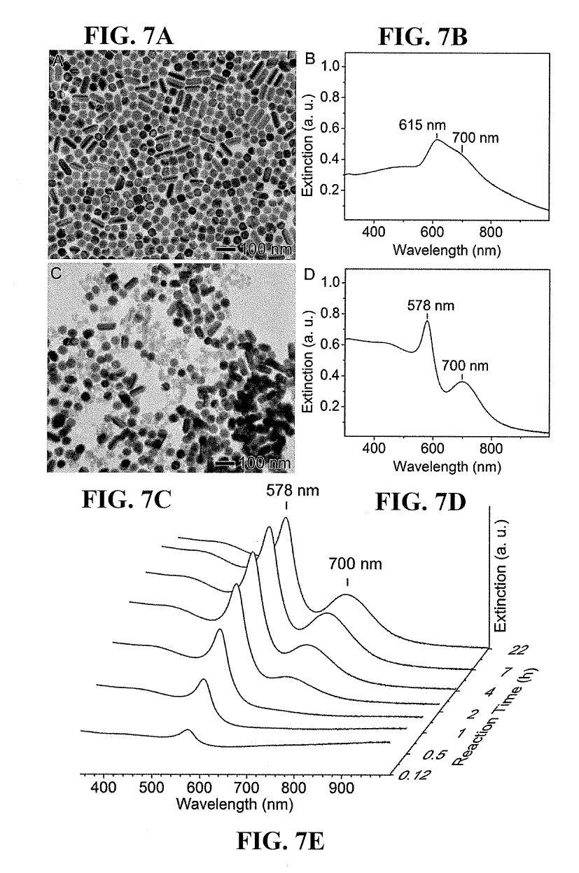

FIGS. 7A and 7B include characterizations of an as-synthesized mixture of Cu nanocubes and nanorods in toluene. For example, FIG. 7A is a TEM image and FIG. 7B is the UV-vis spectrum. FIGS. 7C and 7D are characterizations of the mixture sample after the SiO₂ coating process. FIG. 7E is the spectral evolution of the mixture over time during the SiO₂ coating process.

This example shows that the TOP-mediated sol-gel process in W/O microemulsion can be used to break apart particle assemblies and allow good dispersity of individual nanoparticles in aqueous solution using the SiO₂ coating as a physical barrier.

The feasibility of using the microemulsion method to coat different nanostructures in a mixture was also studied. When formic acid was added to the reaction described in Example 1 above at a lower temperature, a mixture of Cu nanocubes and nanorods was produced. From the TEM image shown in FIG. 7A, the yield of nanorods in the product was about 10-20%. The UV-vis spectrum shows a broad peak at 615 nm with a shoulder at 700 nm (FIG. 7B) which is similar to the spectrum of nanoparticle agglomerates (see e.g., FIG. 5A). The particle suspension having nanorods and nanocubes was then added into hexane, followed by the addition of Igepal CO-520, TEOS, and NaOH solution to create a microemulsion environment for the sol-gel process. After the sol-gel process, the nanostructures were coated by SiO₂ as seen in the TEM image of FIG. 7C and well-dispersed in aqueous solution. As predicted, the UV-vis spectrum of the sample in water exhibits two distinct peaks at 578 nm and 700 nm, see FIG. 7D, which corresponds to the rounded nanocubes and nanorods, respectively. When the reaction was monitored via UV-vis spectroscopy as illustrated in FIG. 7E, the extinction peaks of the nanocubes and nanorods distinctly arise as the coating process proceeds. During the coating process, the peak at 700 nm was gradually recovered while the peak at 615 nm shifted to the blue, indicating that the nanostructure agglomerates were separated to yield individually coated nanocube and nanorod nanostructures. After coating with SiO₂, the nanorods could be separated from the nanocubes via centrifugation because of the difference in mass.

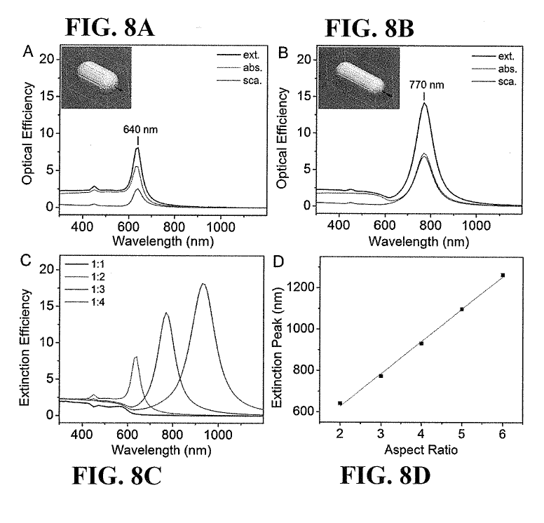

FIGS. 8A-8D is simulated optical spectra data (i.e., analyzed by DDA simulations) associated with Cu nanorods having two different aspect ratios suspended in water. FIG. 8A is for nanorods having a W×L of 37×74 nm and FIG. 8B is for nanorods having a W×L of 37×112 nm. FIG. 8C is the extinction spectra of Cu nanorods having a fixed width of 37 nm but different aspects: 1:1, 2:1, 3:1, and 4:1. FIG. 8D is the position of the extinction peak as a function of the aspect ratio for the Cu nanorods, which shows excellent linearity. The linear line (y=313.8+156.8×, R²=0.997) plotted in FIG. 8D is a linear least square fit of the simulated peak positions.

As FIGS. 8A and 8B illustrate, nanorods having aspect ratios of 2:1 and 3:1 possess extinction peaks at 640 nm and 770 nm, respectively. The relative ratio of absorption to scattering was compared based on the optical efficiency, which is referred to as the ratio of the optical cross-section to the physical cross-section of a nanoparticle. The efficiency of absorption is twice that of scattering for the nanorod with aspect ratio of 2:1, but becomes comparable to the scattering efficiency for the nanorod with aspect ratio of 3:1.

FIG. 8C shows the extinction peaks of nanospheres (i.e., an aspect ratio of 1:1) and nanorods having aspect ratios from 2:1 to 3:1 and 4:1. The peaks respectively increase with increasing aspect ratio from 560 nm to 641, 773, and 931 nm. The extinction peaks of nanorods further increase to 1097 and 1263 nm for aspect ratios of 5:1 and 6:1, respectively. The peak positions for the extinction spectra are proportional to aspect ratios of the nanorods, as plotted in FIG. 8D. A linear least square fitting of the peak position and the aspect ratio gives a Pearson R² of 0.997 showing good linearity (y=313.8+156.8×). This good fit allows the aspect ratio of the synthesized nanorods to be determined by examining the position of the LSPR maximum. UV-vis illustrates the sharp peak at 700 nm, which is consistent with an aspect ratio of 2.5:1. This result agreed well with the aspect ratio of 2.5 estimated from analyzing the TEM image.

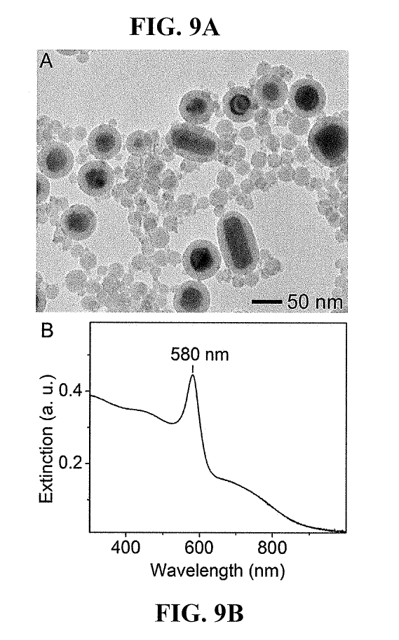

FIGS. 9A and 9B is characterization data for associated with SiO₂ coated Cu nanoparticles having a shell thickness of about 13 nm. FIG. 9A is a TEM image and FIG. 9B is the UV-vis spectrum of the SiO₂ coated Cu nanoparticles having the shell thickness of about 13 nm.

As observed during the core-shell nanoparticle synthesis, the SiO₂ shell thickness can vary based upon the reaction time of the sol-gel process. In studies, the sol-gel process is typically allowed to proceed for 24 h, yielding a shell thickness of about 5 nm. Shell thicknesses of less than 5 nm were also demonstrated but difficult to image. Alternatively, the core-shell nanoparticles could be treated by 0.3 mM FeCl₃ for 10 min to etch the Cu cores, resulting in hollow SiO₂ shell for imaging. Thicker shells can also be formed by increasing the sol-gel reaction time. FIG. 9A is a TEM image of Cu—SiO₂ core-shell nanoparticles having a shell thickness of about 13 nm produced from the sol-gel reaction which was allowed to proceed for 72 h. The UV-vis spectrum of the sample in aqueous solution depicts an extinction at 580 nm as seen in FIG. 9B, which is similar to that of sample having thinner shells. Increasing the shell thickness does not appear to have a significant effect on the LSPR peak position, suggesting that there is little change in the refractive index of the medium. The insensitivity of the peak position to shell thickness implies that the SiO₂ shells are likely porous and filled with water; thus the refractive index of the surrounding medium is similar to that of the water.

Based on the foregoing series of characterization results, it can be concluded that introducing organic capping molecules (e.g., trioctylphosphine (TOP)) to Cu cores prior to the sol-gel reaction that at least partially, and in some embodiments, fully encapsulates the cores in SiO₂ yields a stable aqueous Cu—SiO₂ core-shell suspension in which the LSPR properties of the Cu cores will be well-preserved. With the TOP capping, the oxidation of the Cu cores in the microemulsion was significantly reduced, thus allowing the Cu cores to sustain the sol-gel process used for coating the SiO₂ protection layer. It was found that the self-assembled TOP-capped Cu nanoparticles were spontaneously disassembled during the sol-gel reaction, thus recovering the LSPR of individual particles. During the disassembling progress, the extinction spectrum of the nanocube agglomerates evolved from a broad extinction profile to a desirable sharp, narrow peak. For a mixture of nanocubes and nanorods, the spectra evolved to two distinct peaks during the dissembling process. The observed spectra match well with the numerical simulations. Such Cu—SiO₂ core-shell nanoparticles having sharp and stable LSPR can greatly expand the utilization of Cu nanoparticles in aqueous environments.

Further, it is has been demonstrated that the oxidation resistant Cu—SiO₂ nanoparticles can be solvated easily by water or other polar solvents (e.g., amides, alcohols, amines, keytones (aldehydes), esters, alkyl halides, ethers, aromatics) useful for a broad range of applications. For example, the Cu—SiO₂ core-shell nanoparticles set forth herein can be used as replacements for Au and/or Ag nanoparticles used for optical sensing, antimicrobial applications, biological applications, as well as serving as a superior heterogeneous Cu catalyst for chemical systems where high surface area water soluble Cu is called for.

It can further be concluded in view of the various data collected, that the described compositions and methods enables the production of Cu—SiO₂ core-shell nanoparticles consisting of Cu cores approximately 30 nm in diameter, SiO₂ shells 10 nm or less in thickness, and nanoparticles that are easily dispersed in water. Notably, the choice of TOP as the nanoparticle capping agent and the sol-gel catalyst (e.g., NaOH or KOH) chosen to employ during the SiO₂ encapsulation process contribute to the high quality of the resultant Cu—SiO₂ core shell nanoparticles. Bases, such as ammonium hydroxide or tetrabutylammonium hydroxide, which are commonly used as catalysts in synthesis reactions, will actually destroy the Cu particles. As the data concluded herein, the Cu—SiO₂ core-shell nanoparticles provided herein undergo no observable change when dispersed in water at ambient conditions for at least one month, can be indefinitely preserved by freezing or freeze drying, and can be dispersed in water at a later date with the properties intact.

Since the particles herein resist oxidation, even when dissolved in water, they retain their conductive properties, allowing them to replace Au—SiO₂ and Ag—SiO₂ core-shell nanoparticles in many applications. Due to the lower cost compared with Au and Ag, Cu is naturally attractive as a cost saving alternative to the noble metals. In addition to replacing Au—SiO₂ and Ag—SiO₂ nanoparticles in applications requiring their optical properties, such as color engineering, chemical sensing, and plasmonics, these Cu—SiO₂ nanoparticles can also be used for their distinct chemical properties arising from their metallic Cu core, in applications such as catalysis, photothermal agents, tribology fillers, and antimicrobials. Furthermore, when used as a catalyst, they are excellently dissolved in polar solvents during the chemical reaction, and can be separated using common techniques to recover and re-use the nanoparticles.

As described herein, a TOP-mediated, sol-gel process in a microemulsion environment successfully produces Cu—SiO₂ core-shell nanoparticles that can be well dispersed in aqueous solutions, including water. The SiO₂ coating acts as a protective layer to prevent oxidation of the Cu cores, thus preserving the superior LSPR properties of Cu cores even in the aqueous solution. Cu—SiO₂ core-shell nanoparticles having substantially cubic cores, including rounded cubic cores, exhibit a narrow (i.e., sharp) and intense LSPR peak at around 590 nm, or a subrange from 560-600 nm, while the Cu—SiO₂ core-shell nanoparticles having nanorod Cu cores exhibit an LSPR peak at around 700 nm, and having aspect ratio of approximately 2.5:1.

Unlike spherical nanoparticles, the dampening from the interband transitions of Cu was significantly weakened for the nanocubes and nanorods due to the shape effect. Aqueous Cu—SiO₂ core-shell nanostructures can be used for sensing, catalysis, fillers, photothermal agents, and/or antimicrobial applications. The ligand-mediated (e.g., TOP mediated) sol-gel method provides a versatile approach to spontaneously disperse ligand-induced nanoparticle agglomerates and recover the individual particle dispersion for various applications in aqueous environments.

The compositions and methods set forth herein provide improved Cu—SiO₂ core-shell nanoparticles that can easily dispersed in water and have a long shelf life. The Cu—SiO₂ nanoparticles are improved in terms of uniformity of shape, size, and shell thickness. The particles can be preserved as a powder or frozen in water, analogous to commercial proteins, and will retain their properties for at least one month after rehydration, and in some embodiments, between 1-6 months. The Cu—SiO₂ nanoparticles described herein can be used in place of gold and silver nanoparticles in applications where nanosized optical properties are required, as well as chemical systems requiring water soluble Cu, with the advantage of extremely high surface area, reducing required materials and cost.

Various embodiments of the present subject matter have been described in fulfillment of the various objectives set forth above. It should be recognized that these embodiments are merely illustrative of the principles of the present invention. Numerous modifications and adaptations thereof will be readily apparent to those skilled in the art without departing from the scope of the instant disclosure.

1. A nanoparticle composition comprising:
core-shell nanoparticles comprising copper (Cu) core components and silica (SiO₂) shell components encapsulating the copper core components.

2. The nanoparticle composition of claim 1, wherein the core-shell nanoparticles have an average particle size of 10-60 nanometers (nm).

3. The nanoparticle composition of claim 1, wherein the core components have an average core diameter of 20-40 nanometers (nm).

4. The nanoparticle composition of claim 1, wherein the shell components have an average shell thickness of 20 nanometers (nm) or less.

5. The nanoparticle composition of claim 1, wherein the core components are non-spherical.

6. The nanoparticle composition of claim 5, wherein the core components are cubes, rounded cubes, nanorods, or combinations thereof.

7. The nanoparticle composition of claim 1, wherein an outer surface of each core component is passivated.

8. The nanoparticle composition of claim 1, further comprising an aqueous continuous phase in which the core-shell nanoparticles are dispersed.

9. The nanoparticle composition of claim 1, wherein the aqueous continuous phase consists essentially of water.

10. The nanoparticle composition of claim 1, wherein the composition exhibits an optical spectrum having a localized surface plasmon resonance (LSPR) peak in the visible spectral range.

11. The nanoparticle composition of claim 10, wherein the LSPR peak ranges from 560-600 nm.

12. The nanoparticle composition of claim 11, wherein the LSPR peak ranges from 560-600 nm after being stored for at least one month.

13. The nanoparticle composition of claim 1, wherein the composition exhibits an optical spectrum having two localized surface plasmon resonance (LSPR) peaks in the visible spectral range.

14. The nanoparticle composition of claim 13, wherein a first LSPR peak ranges from 560-595 nm and a second LSPR peak ranges from 600-800 nm.

15. The nanoparticle composition of claim 1, further comprising an antimicrobial.

16. The nanoparticle composition of claim 1, further comprising a heterogeneous catalyst.

17. The nanoparticle composition of claim 1, further comprising a photothermal agent.

18. The nanoparticle composition of claim 1, further comprising a tribology filler or tribology composition.

19. The nanoparticle composition of claim 1, further comprising a detection reagent for a Surface Enhanced Raman Spectroscopy (SERS) system or a SERS detector.

20. The nanoparticle composition of claim 1, further comprising a medical imaging contrast agent.

21. A method of making a composition comprising:
providing a population of copper (Cu) nanoparticles having organic capping molecules adsorbed onto surfaces thereof; and
forming silica (SiO₂) shells over the nanoparticles.

22. The method of claim 21, wherein providing a population of Cu nanoparticles comprises providing a population of preformed Cu nanoparticles.

23. The method of claim 22, wherein forming SiO₂ shells over the nanoparticles comprises condensing SiO₂ onto a surface of the preformed Cu nanoparticles.

24. The method of claim 21, further comprising dispersing the nanoparticles in an aqueous solution.

25. The method of claim 24, further comprising storing the nanoparticles in the aqueous solution for one week, two weeks, one month, or less than six months.

26. The method of claim 21, wherein the Cu nanoparticles have an average size of 20-40 nanometers (nm).

27. The method of claim 21, wherein the SiO₂ shells have an average shell thickness of 20 nanometers (nm) or less.

28. The method of claim 21, wherein the Cu nanoparticles are cubes, rounded cubes, nanorods, or combinations thereof.

29. A population of core-shell nanoparticles comprising the reaction product of:
copper (Cu) nanoparticles having a particle size of 20 to 40 nm and organic capping molecules adsorbed onto surfaces of the Cu nanoparticles; and
a microemulsion formed from a hydrophobic solvent, water, a silicon (Si) containing compound, and an alkali hydroxide catalyst.

30. The nanoparticles of claim 29, wherein the organic capping molecules are trioctylphosphine (TOP).

31. The nanoparticles of claim 29, wherein the alkali hydroxide catalyst is sodium hydroxide (NaOH) or potassium hydroxide (KOH).

32. The nanoparticles of claim 29, wherein the microemulsion is devoid of ammonia.
